SEQUENCE LISTING (27 sequences)

SEQ ID NO: 1 - protein, 378 aa, synthetic construct
  REGION 1..378: note = synthetic sequence
LQLDASTVYAVGRGDSPASSAVPGIGVPGIGVPGKGVPGIGVPGIGVPGIGVPGIGVPGKGVPGIGVPGIGVPGIGVPGIGVPGKGVPGIGVPGIGLDASTVYAVGRGDSPASSAVPGIGVPGIGVPGKGVPGIGVPGIGVPGIGVPGIGVPGKGVPGIGVPGIGVPGIGVPGIGVPGKGVPGIGVPGIGLDASTVYAVGRGDSPASSAVPGIGVPGIGVPGKGVPGIGVPGIGVPGIGVPGIGVPGKGVPGIGVPGIGVPGIGVPGIGVPGKGVPGIGVPGIGLDASTVYAVGRGDSPASSAVPGIGVPGIGVPGKGVPGIGVPGIGVPGIGVPGIGVPGKGVPGIGVPGIGVPGIGVPGIGVPGKGVPGIGVPGIG

SEQ ID NO: 2 - protein, 378 aa, synthetic construct
  REGION 1..378: note = synthetic sequence
LQLDASTVYAVGRDGSPASSAVPGIGVPGIGVPGKGVPGIGVPGIGVPGIGVPGIGVPGKGVPGIGVPGIGVPGIGVPGIGVPGKGVPGIGVPGIGLDASTVYAVGRDGSPASSAVPGIGVPGIGVPGKGVPGIGVPGIGVPGIGVPGIGVPGKGVPGIGVPGIGVPGIGVPGIGVPGKGVPGIGVPGIGLDASTVYAVGRDGSPASSAVPGIGVPGIGVPGKGVPGIGVPGIGVPGIGVPGIGVPGKGVPGIGVPGIGVPGIGVPGIGVPGKGVPGIGVPGIGLDASTVYAVGRDGSPASSAVPGIGVPGIGVPGKGVPGIGVPGIGVPGIGVPGIGVPGKGVPGIGVPGIGVPGIGVPGIGVPGKGVPGIGVPGIG

SEQ ID NO: 3 - protein, 4 aa, synthetic construct
  REGION 1..4: note = synthetic sequence
DGEA

SEQ ID NO: 4 - protein, 12 aa, synthetic construct
  REGION 1..12: note = synthetic sequence
PLAEIDGIELTY

SEQ ID NO: 5 - protein, 8 aa, synthetic construct
  REGION 1..8: note = synthetic sequence
VFDNFVLK

SEQ ID NO: 6 - protein, 5 aa, synthetic construct
  REGION 1..5: note = synthetic sequence
IKVAV

SEQ ID NO: 7 - protein, 5 aa, synthetic construct
  REGION 1..5: note = synthetic sequence
YIGSR

SEQ ID NO: 8 - protein, 5 aa, synthetic construct
  REGION 1..5: note = synthetic sequence
HAVDI

SEQ ID NO: 9 - protein, 10 aa, synthetic construct
  REGION 1..10: note = synthetic sequence
HAVDIHAVDI

SEQ ID NO: 10 - protein, 17 aa, synthetic construct
  REGION 1..17: note = synthetic sequence
TVYAVTGRGDSPASSAA

SEQ ID NO: 11 - protein, 20 aa, synthetic construct
  REGION 1..20: note = synthetic sequence
VSDPGYIGSRSDDSASASAA

SEQ ID NO: 12 - protein, 16 aa, synthetic construct
  REGION 1..16: note = synthetic sequence
VGPAGGDGEAGAQGPP

SEQ ID NO: 13 - protein, 28 aa, synthetic construct
  REGION 1..28: note = synthetic sequence
SGSGGSGGPLAEIDGIELTYGGSGGSGS

SEQ ID NO: 14 - protein, 26 aa, synthetic construct
  REGION 1..26: note = synthetic sequence
SGSGGSGGLDVFDNFVLKGGSGGSGS

SEQ ID NO: 15 - protein, 25 aa, synthetic construct
  REGION 1..25: note = synthetic sequence
SGSGGSGGLDVFDNFVLGGSGGSGS

SEQ ID NO: 16 - protein, 21 aa, synthetic construct
  REGION 1..21: note = synthetic sequence
SGSGGSGGHAVDIGGSGGSGS

SEQ ID NO: 17 - protein, 28 aa, synthetic construct
  REGION 1..28: note = synthetic sequence
SGSGGSGGHAVDINGHAVDIGGSGGSGS

SEQ ID NO: 18 - protein, 21 aa, synthetic construct
  REGION 1..21: note = synthetic sequence
SGSGGSGGADHIVGGSGGSGS

SEQ ID NO: 19 - protein, 17 aa, synthetic construct
  REGION 1..17: note = synthetic sequence
TVYAVTGRDGSPASSAA

SEQ ID NO: 20 - protein, 25 aa, synthetic construct
  REGION 1..25: note = synthetic sequence
VPGIGVPGIGVPGKGVPGIGVPGIG

SEQ ID NO: 21 - protein, 30 aa, synthetic construct
  REGION 1..30: note = synthetic sequence
VPGIGVPGKGVPGYGVPGIGVPGKGVPGIG

SEQ ID NO: 22 - protein, 19 aa, synthetic construct
  REGION 1..19: note = synthetic sequence
ARKQAASIKVAVSADRASA

SEQ ID NO: 23 - protein, 5 aa, synthetic construct
GGGGS

SEQ ID NO: 24 - protein, 14 aa, synthetic construct
GSTSGSGKSSEGKG

SEQ ID NO: 25 - protein, 5 aa, synthetic construct
VPGIG

SEQ ID NO: 26 - protein, 5 aa, synthetic construct
VPGKG

SEQ ID NO: 27 - protein, 5 aa, synthetic construct
VPGYG

(19) United States
(12) Patent Application Publication
(10) Pub. No.: US 2024/0150499 A1
(43) Pub. Date: May 9, 2024

(54) DYNAMIC RECOMBINANT HYDROGELS WITH DEGRADATION-INDEPENDENT RELAXATION KINETICS

(71) Applicant: The Board of Trustees of the Leland Stanford Junior University, Stanford, CA (US)

(72) Inventors: Renato Navarro, Menlo Park, CA (US); Michelle Huang, Stanford, CA (US); Julien Roth, Stanford, CA (US); Kelsea Hubka, Stanford, CA (US); Sarah C. Heilshorn, Mountain View, CA (US)

(21) Appl. No.: 18/489,331

(22) Filed: Oct. 18, 2023

Related U.S. Application Data
(60) Provisional application No. 63/380,486, filed on Oct. 21, 2022.

(51) Int. Cl. C08B 37/08; C07K 14/78; C08J 3/075; C12N 5/00

(52) U.S. Cl. CPC C08B 37/0072; C07K 14/78; C08J 3/075; C12N 5/0068; C12N 2533/50; C12N 2533/80; C12N 2537/10

(57) ABSTRACT

A two-component hydrogel matrix system is described, which has a variety of benefits for cell and tissue culture; including without limitation hydrogels that allow for cells to more easily migrate into/through, proliferate, spread, and deposit matrix within the gel; stable hydrogels that allow for higher retention that can be held together for prolonged periods of time, etc. The components comprise: (1) chemically modified hyaluronic acid (HA) comprising an aldehyde or benzaldehyde containing side group, and a bicyclononyne containing side group, and (2) chemically modified elastin-like protein (ELP) comprising a hydrazine containing side group and an azide containing side group.

### CROSS-REFERENCE TO RELATED APPLICATIONS

This application claims the benefit of U.S. Provisional Patent Application No. 63/380,486, filed Oct. 21, 2022, which application is incorporated herein by reference in its entirety.

### INCORPORATION BY REFERENCE OF SEQUENCE LISTING

A Sequence Listing is provided herewith as a Sequence Listing XML, “STAN-1989_Sequence_Listing” created on Oct. 16, 2023, and having a size of 33,430 bytes. The contents of the Sequence Listing XML are incorporated herein by reference in their entirety.

### BACKGROUND

Viscoelasticity is a desirable material property for biomaterials, as it allows for many gels to be injectable and for cells to more easily migrate into/through, proliferate, spread, and deposit matrix within the gel. However, most viscoelastic gels suffer from poor stability because they are prone to rapid gel erosion, since the crosslinks within the polymer network are dynamic and reversible. Elastic gels offer high stability because they have static covalent crosslinks with the polymer network, however, do not have stress relaxation properties. There is a growing need for viscoelastic gels that have both high stability while also exhibiting stress relaxation properties. Provided herein are systems and kits for generation of viscoelastic hydrogels that are both stable and exhibit stress relaxation properties.

### SUMMARY

A two-component hydrogel matrix system is described, which has a variety of benefits for cell and tissue culture. Included are hydrogels that allow for cells to more easily migrate into the gel, through the gel, proliferate, spread, and deposit matrix within the gel. Provided are stable hydrogels that allow for higher retention that can be held together for prolonged periods of time. The components comprise: (1) chemically modified hyaluronic acid (HA) comprising an aldehyde or benzaldehyde containing side group and comprising a bicyclononyne containing side group, and (2) chemically modified elastin-like protein (ELP) comprising a hydrazine containing side group and an azide containing side group. Mixing the modified biopolymers together induces the formation of dynamic bonds between the aldehyde or benzaldehyde containing side group and the hydrazine containing side group (also known as hydrazone bonds); and formation of static bonds between the bicyclononyne containing side group and the azide containing side group (referred to herein as spot welds, and an example from the class of reactions known as strain-promoted azide-alkyne cycloadditions) resulting in the formation of a hydrogel network. Selection of the ratio between (1) and (2) allows tuning of critical variables of the hydrogel, including, for example, matrix stiffness, matrix stress relaxation rate, and cell-adhesive-ligand concentration and identity. These variables can be independently and quantitatively defined. Mixing of (1) and (2) into a hydrogel formulation is here termed a HELP (hyaluronic acid elastin-like protein) hydrogel.

The hyaluronic acid component is chemically modified to comprise a benzaldehyde or aldehyde side group, and a bicyclononyne containing side group. The specific ratio of the benzaldehyde and the aldehyde side groups in the final hydrogel formulation controls the stress relaxation variable. A key feature of native extracellular matrices is their ability to undergo stress relaxation due to their physical crosslinks, which can be easily remodeled. Compositions comprising a greater percentage of hyaluronic acid modified with an aldehyde containing functional group increase the average kinetic exchange rate of the gel, leading to a faster stress-relaxation rate. A greater percentage of hyaluronic acid modified with a benzaldehyde group decreases the average kinetic exchange rate of the gel, leading to slower stress-relaxation rate. Modifications to stress-relaxation rate can be achieved independently of matrix ligand composition and stiffness. The ratio of HA-benzaldehyde to HA-aldehyde may be pre-selected for a hydrogel of interest, usually ranging from about 100:0 to 0:100, for example at a ratio from about 95:5, 90:10, 75:25, 50:50, 25:75, etc.

The ELP component comprises a recombinant sequence of elastin-like sequences optionally interspersed with cell-adhesive sequences. To engage in crosslinking with chemically modified HA, the ELP is chemically modified to comprise a hydrazine group and an azide group. The cell-adhesive sequence within the ELP may be selected from an integrin-binding, fibronectin-based, extended RGD sequence, a scrambled RGD sequence, a cell-adhesive sequence derived from collagen type I, e.g. (SEQ ID NO:3) DGEA, a cell adhesive sequence derived from tenascin, e.g. (SEQ ID NO:4) PLAEIDGIELTY, (SEQ ID NO:5) VFDNFVLK, etc.; a cell adhesive sequence derived from laminin, e.g. (SEQ ID NO:6) IKVAV, (SEQ ID NO:7) YIGSR, etc.; a cell adhesive sequence derived from cadherin, e.g. (SEQ ID NO:8) HAVDI, (SEQ ID NO:9) HAVDIHAVDI; and the like. For example, SEQ ID NO:1 and SEQ ID NO:2 are ELPs with an RGD sequence, and a scrambled RGD sequence, respectively.

The cell-adhesive sequence concentration of the hydrogel can be varied by adjusting the ratio of ELP comprising an RGD motif, to ELP lacking an RGD motif or comprising scrambled or non-RGD cell-adhesive motifs, as disclosed above. The ratio may be pre-selected for a hydrogel of interest, usually ranging from about 100:0 to 0:100, for example from about 75:25, 50:50, 25:75, 10:90. For some applications, a HELP hydrogel comprises from about 0.25 mM RGD up to about 1.5 mM RGD, e.g. about 0.25 mM, 0.5 mM, 0.75 mM, 1 mM, 1.25 mM, 1.5 mM.

Matrix stiffness is determined by the concentration and ratio of hydrazine to aldehyde and benzaldehyde side groups (dynamic bonds), and by the concentration and ratio of bicyclononyne to azide side groups (static bonds). The ratio of the hydrazine to aldehyde and benzaldehyde side groups may be varied, e.g. from about 1:3, about 1:2, about 1.5:1, about 1.25:1, about 1:1, about 1:1.25, about 1:1.5, about 1:2, about 1:3, etc. The ratio of the bicyclononyne to azide side groups may be varied, e.g. from about 1:3, about 1:2, about 1.5:1, about 1.25:1, about 1:1, about 1:1.25, about 1:1.5, about 1:2, about 1:3, etc. The ratio of the dynamic bonds to static bonds may have an impact on the matrix stiffness or may not alter matrix stiffness depending on the ratio of dynamic to static bonds. The ratio of dynamic to static bonds may be in a range that is at least about 30:1, about 14:1, about 7:1, about 5:1, to at least about 3:1. Depending on the specific ratio of dynamic to static bonds and the overall concentration of dynamic and static bonds, the matrix stiffness (shear storage modulus) may vary from at least about 100 Pa to at least about 15 kPa. The ratio of dynamic bonds and static bonds can be altered through three variables: (1) number of hydrazine and azide groups per ELP molecule, (2) number of aldehyde or benzaldehyde and bicyclononyne groups per HA molecule, and (3) the blending of the chemically modified ELP and chemically modified HA.

The presence of static bonds within a hydrogel matrix system comprising dynamic bonds can provide a number of benefits. In some embodiments, the presence of static bonds within a hydrogel matrix system comprising dynamic bonds does not affect the overall stiffness of the hydrogel matrix system as compared to a hydrogel matrix system comprising only dynamic bonds, while increasing the stability of the hydrogel matrix system over time. In some embodiments, the presence of static bonds within a hydrogel matrix system comprising dynamic bonds does not inhibit the shear thinning behavior of the hydrogel matrix system as compared to a hydrogel matrix system comprising only dynamic bonds, while increasing the stability of the hydrogel matrix system over time.

The presence of static bonds within a hydrogel matrix system comprising dynamic bonds increases the stability of the hydrogel matrix system. The stability of the hydrogel matrix system can be measured as the resistance or delay of erosion of the hydrogel matrix system. The erosion of the hydrogel matrix system is measured by the amount of ELP polymer remaining in the hydrogel matrix system at a given time point. The presence of static bonds within a hydrogel matrix system comprising dynamic bonds can increase the amount of time that the hydrogel matrix system is able to retain at least 60% of the ELP within the hydrogel matrix system. The amount of time may be a range of different times. For instance, the presence of static bonds within a hydrogel matrix system comprising dynamic bonds may retain at least 60% of the ELP within the hydrogel matrix system from at least about 3 days to at least about 30 days.

In another aspect of the invention, the matrix is extruded through a syringe needle or a catheter or a bioprinter to form a solid structure either in air or within a printing support bath. The solid structure maintains its structural stability after release from the printing support bath. After extrusion, the solid structure maintains its structural stability. Cells encapsulated within this matrix can be extruded in this manner for use as a bio-ink for 3D bioprinting; for injectable regenerative medicine therapy; etc. A shear-thinning material with a fracture stress below 2,000 Pa is injectable by hand force. The fracture stress can be adjusted by changing three variables: (1) the molecular weight of the HA, usually with MW below 100 kDa, (2) the kinetics of the hydrazone bond, with the fast exchange kinetics of the hydrazine-aldehyde preferred over the slower exchange kinetics of the hydrazine-benzaldehyde reaction for this purpose, and (3) the overall polymer concentration, usually with a final concentration of from about 0.5-3 wt % of ELP and from about 0.5-3 wt % of HA.

### BRIEF DESCRIPTION OF THE DRAWINGS

FIG. 1 is a schematic depiction of the differences between gels with only static bonds, only dynamic bonds and a combination of static and dynamic bonds.

FIG. 2 is a schematic depiction of exemplar elastin-like protein (ELP) , with SEQ ID NO:10 and SEQ ID NO:20; and hyaluronic acid (HA) compositions with chemical modifications to achieve a combination of static and dynamic bonds.

FIG. 3 is a schematic depiction of an exemplary hydrogel matrix system with a combination of static and dynamic bonds.

FIG. 4 depicts gelation time of an exemplary hydrogel matrix system.

FIG. 5 depicts shear storage modulus of an exemplary hydrogel matrix system.

FIG. 6 depicts stress relaxation of an exemplary hydrogel matrix system.

FIG. 7 depicts the degradation profile of hydrogel matrix systems with and without static bonds (spot-welds).

FIG. 8 depicts the structural stability of an exemplary hydrogel matrix system upon extrusion printing with a 3D bioprinter before and after release from a support bath.

FIG. 9 is a schematic depiction of exemplar elastin-like protein (ELP) bioconjugation, with SEQ ID NO:10 and SEQ ID NO:20.

FIG. 10 is a schematic depiction of exemplar hyaluronic acid (HA) bioconjugation.

### DETAILED DESCRIPTION

### Definitions

Before embodiments of the present disclosure are further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of embodiments of the present disclosure.

It must be noted that as used herein and in the appended claims, the singular forms “a”, “and”, and “the” include plural referents unless the context clearly dictates otherwise. Thus, for example, reference to “a compound” includes not only a single compound but also a combination of two or more compounds, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

As used herein, the phrases “for example,” “for instance,” “such as,” or “including” are meant to introduce examples that further clarify more general subject matter. These examples are provided only as an aid for understanding the disclosure, and are not meant to be limiting in any fashion.

The term “hydrogel” is used in its conventional sense to refer to a material that absorbs a solvent (e.g. water), undergoes swelling without measurable dissolution, and maintains three-dimensional networks capable of reversible deformation. “Swelling” as referred to herein is meant the isotropic expansion of the hydrogel structure as water molecules diffuse throughout the internal volume of the hydrogel. The properties of copolymer hydrogels disclosed herein may be modulated as desired, by varying the amounts of each component, ratios of each component or the density of specific components, as described in greater detail below. The term hydrogel may include both desiccated and hydrated (e.g., solvent swollen) hydrogels.

In some embodiments of the invention a hydrogel provides a scaffold for cell growth, including growth of metabolically active cells, e.g. differentiating cells, etc. The cells may be grown in vitro, e.g. a culture of one or a plurality of cell types. Cells may also be grown in vivo, e.g. where a hydrogel provides a substrate for regenerative cell growth. The hydrogels used in the invention provide appropriate mechanical strength for long term structural stability.

An Elastin-like Protein (ELP) comprises a recombinant sequence of elastin-like sequences optionally interspersed with cell-adhesive sequences. To engage in crosslinking with chemically modified HA, the ELP is chemically modified to comprise a hydrazine group. The optional cell-adhesive sequence within the ELP comprises a motif involved in cell adhesion, which may be selected from an integrin-binding, fibronectin-based, extended RGD sequence, a scrambled RGD sequence, a cell-adhesive sequence derived from collagen type I, e.g. (SEQ ID NO:3) DGEA, a cell adhesive sequence derived from tenascin, e.g. (SEQ ID NO:4) PLAEIDGIELTY, (SEQ ID NO:5) VFDNFVLK, etc.; a cell adhesive sequence derived from laminin, e.g. (SEQ ID NO:6) IKVAV, (SEQ ID NO:7) YIGSR, etc.; a cell adhesive sequence derived from cadherin, e.g. (SEQ ID NO:8) HAVDI, (SEQ ID NO:9) HAVDIHAVDI; and the like.

The cell-adhesive domain of the engineered elastin-like protein can be designed to include alternative peptide-sequences known to interact with cell-surface receptors. These sequences can include peptides derived from native extracellular matrix proteins (e.g. fibronectin, laminin, collagen, tenascin-C) or peptides derived from cell-cell adhesion receptors (e.g. N-cadherin) (Table 1). Selection of the cell adhesive peptide sequence together with the elastin-like region sequence defines the overall hydrophobicity of the engineered protein, and hence controls the lower critical solution temperature (LCST) behavior.

In some embodiments an ELP comprises the structure:

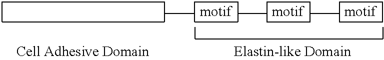

where the cell adhesive domain is from about 15 to about 45 amino acids in length and comprises one or more cell adhesion sequence motifs, which may be selected from RGD, scrambled RGD, no RGD, or any of SEQ ID NO:3 to SEQ ID NO:9. SEQ ID NO:10-19 and 22 are exemplary.

Linker sequences optionally flank the cell adhesion sequence motif, where a peptide linker can be between about 5 to 20, 5 to 15, 5 to 10 or 5 to 9 amino acids in length. Exemplary linkers include linear peptides having at least two amino acid residues such as Gly-Gly, Gly-Ala-Gly, Gly-Pro-Ala, GGGGS (SEQ ID NO:23), or multiples thereof. Suitable linear peptides include poly glycine, polyserine, polyproline, polyalanine and oligopeptides consisting of alanyl and/or serinyl and/or prolinyl and/or glycyl amino acid residues. In one embodiment a linker comprises the amino acid sequence GSTSGSGKSSEGKG (SEQ ID NO:24), or (GGGGS)n (SEQ ID NO:23), where n is 1, 2, 3, 4, 5, etc.; however many such linkers are known and used in the art and may serve this purpose.

The elastin-like domain is comprised of elastin-like motifs, which include, without limitation, (SEQ ID NO:25) VPGIG; (SEQ ID NO:26) VPGKG; (SEQ ID NO:27) VPGYG. One or more of SEQ ID NO:25, 26, 27 can be present in a protein. In some embodiments the number of motifs is from 1 to 7, from 1 to 6, from 2 to 5, from 3 to 5; and may be about 5 motifs. Exemplary domain sequences are provided in, for example, SEQ ID NO:20 and 21. Examples include, without limitation, SEQ ID NO:1, LQ(LDASTVYAVGRGDSPASSA[(VPGIG)₂VPGKG(VPGIG)₂]₃)₄ and SEQ ID NO:2, LQ(LDASTVYAVGRDGSPASSA[(VPGIG)₂VPGKG(VPGIG)₂]₃)₄.

The ELP protein is chemically modified to comprise a hydrazine group, and may comprise from about 3 to about 20 hydrazine groups, from about 5 to about 18, from about 10 to about 14 groups. The ELP protein is also modified to comprise azide groups, in a ratio appropriate for the number of hydrazines, e.g. which may be present at from about 1% to about 15% of the available groups, for example from about 1% to about 10%, from about 1% to about 5%, from about 5% to about 15%. Standard bioconjugation chemistry can be used to attach azide and hydrazines at sites of any of lysine, cysteine, or tyrosine amino acids.

Hyaluronic acid is an anionic, non-sulfated glycosaminoglycan distributed widely throughout connective, epithelial, and neural tissues. Hyaluronic acid is a polymer of disaccharides, themselves composed of D-glucuronic acid and N-acetyl-D-glucosamine, linked via alternating β-(1→4) and β-(1→3) glycosidic bonds. Hyaluronic acid can be up to 25,000 disaccharide repeats in length. Polymers of hyaluronic acid can range in size from about 20 kDa to about 1.5 MDa; from about 20 kDa to about 1 MDa.

The hyaluronic acid is chemically modified to comprise a benzaldehyde or aldehyde side group. The HA is usually modified at from about 5% to about 40% of the available reactive groups, and may be from about 7% to about 20%, from about 10% to about 15%, from about 20% to about 40% and may be around 12% modified. The hyaluronic acid is also modified to comprise bicyclononyne groups, which may be present at from about 1% to about 15% of the available groups, for example from about 1% to about 10%, from about 1% to about 5%, from about 5% to about 15%.

For an aldehyde functional group the carboxylic acid groups on HA are amidated with propargylamine, generating an HA-alkyne intermediate; then, copper click chemistry was used to react this alkyne with the azide moiety of a heterobifunctional small molecule containing an aldehyde functional group onto the HA, generating HA functionalized with aldehydes.

Benzaldehyde modification can be accomplished by first modifying HA to comprise alkyne groups at a desired concentration, e.g. from about 3% to about 40%. HA-alkynes are then modified with an azide moiety of a heterobifunctional small molecule containing a benzaldehyde functional group to generate HA-benzaldehyde.

The term “hydrazone bond” as used herein refers to the bond formed by the following chemical reaction:

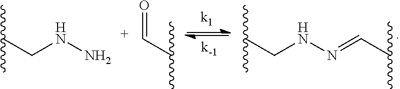

These bonds are referred to as “dynamic” bonds, which dynamic covalent bonds, which can spontaneously break and reform under physiological conditions.

The term “static bond” as used herein refers to a bond formed by the following chemical reaction:

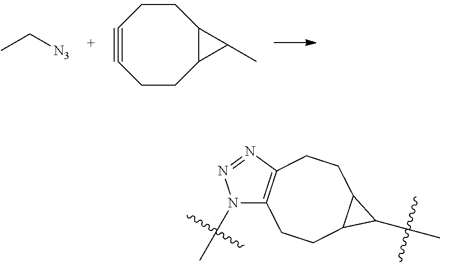

Static bonds are more stable than dynamic bonds under physiological conditions.

The term “storage modulus” as used herein may be used interchangeably with the term “stiffness”. Storage modulus refers to the ratio of the elastic stress to strain and indicates a material's ability to store energy. The storage modulus may be measured using oscillatory shear rheology.

The term “shear thinning” as used herein refers to the specific behavior of a non-Newtonian fluid that loses viscosity as the shear strain increases. In the context of the hydrogel matrix system, shear thinning is the behavior in which the hydrogel matrix undergoes disassembly when a force is applied, e.g. when the hydrogel matrix system is extruded through a syringe, and reassembles in the absence of a force.

As used herein, the terms “treatment,” “treating,” and the like, refer to obtaining a desired pharmacologic and/or physiologic effect, such as reduction of viral titer. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease (e.g., reduction in viral titers).

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; avians, and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “determining,” “measuring,” “assessing,” and “assaying” are used interchangeably and include both quantitative and qualitative determinations.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

A “therapeutically effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of a compound calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

The term “somatic cell” encompasses any cell in an organism that cannot give rise to all types of cells in an organism, i.e. it is not pluripotent. In other words, somatic cells are cells that have differentiated sufficiently that they will not naturally generate cells of all three germ layers of the body, i.e. ectoderm, mesoderm and endoderm.

The term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). A “stem cell” is a cell characterized by the ability of self-renewal through mitotic cell division and the potential to differentiate into a tissue or an organ. Among mammalian stem cells, embryonic and somatic stem cells may be distinguished. Pluripotent stem cells, which include embryonic stem cells, embryonic germ cells and induced pluripotent cells, can contribute to tissues of a prenatal, postnatal or adult organism.

The terms “primary cells”, “primary cell lines”, and “primary cultures” are used interchangeably herein to refer to cells and cell cultures that have been derived from a subject and allowed to grow in vitro for a limited number of passages, i.e. splittings, of the culture. For example primary cultures are cultures that may have been passaged 0 times, 1 time, 2 times, 4 times, 5 times, 10 times, or 15 times, but not enough times go through the crisis stage. Typically, the primary cell lines of the present invention are maintained for fewer than 10 passages in vitro.

The subject cells may be from any mammal, including humans, primates, domestic and farm animals, and zoo, laboratory or pet animals, such as dogs, cats, cattle, horses, sheep, pigs, goats, rabbits, rats, mice etc. They may be established cell lines or they may be primary cells, where “primary cells”, “primary cell lines”, and “primary cultures” are used interchangeably herein to refer to cells and cells cultures that have been derived from a subject and allowed to grow in vitro for a limited number of passages.

The subject cells may be isolated from fresh or frozen cells, which may be from a neonate, a juvenile or an adult, and from tissues including skin, muscle, bone marrow, peripheral blood, umbilical cord blood, spleen, liver, pancreas, lung, intestine, stomach, adipose, and other differentiated tissues. The tissue may be obtained by biopsy or aphoresis from a live donor, or obtained from a dead or dying donor within about 48 hours of death, or freshly frozen tissue, tissue frozen within about 12 hours of death and maintained at below about −20° C., usually at about liquid nitrogen temperature (−190° C.) indefinitely. For isolation of cells from tissue, an appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

The term “cell culture” or “culture” means the maintenance of cells in an artificial, in vitro environment. It is to be understood, however, that the term “cell culture” is a generic term and may be used to encompass the cultivation not only of individual cells, but also of tissues or organs.

Culture conditions of interest provide an environment permissive for differentiation, in which stem or progenitor cells will proliferate, differentiate, or mature in vitro. Such conditions may also be referred to as differentiative conditions. Features of the environment include the medium in which the cells are cultured, any growth factors or differentiation-inducing factors that may be present, and a supporting structure of a hydrogel as disclosed herein. Differentiation may be initiated by formation of organoids, or similar structures.

A “long term culture” used herein refers to a culture in which cells grow, differentiate and are viable for at least about 10 days, or more than 30 days, or more than 60 days, or more than 100 days or more than 150 days.

The term “explant” is used herein to mean a piece of tissue and the cells thereof originating from mammalian tissue that is cultured in vitro, for example according to the methods of the invention. The mammalian tissue from which the explant is derived may obtained from an individual, i.e. a primary explant, or it may be obtained in vitro, e.g. by differentiation of induced pluripotent stem cells.

The term “organoid” is used herein to mean a 3-dimensional growth of mammalian cells in culture that retains characteristics of the tissue in vivo, e.g. prolonged tissue expansion with proliferation, multilineage differentiation, recapitulation of cellular and tissue ultrastructure, etc. A primary organoid is an organoid that is cultured from an explant, i.e. a cultured explant. A secondary organoid is an organoid that is cultured from a subset of cells of a primary organoid, i.e. the primary organoid is fragmented, e.g. by mechanical or chemical means, and the fragments are replated and cultured. A tertiary organoid is an organoid that is cultured from a secondary organoid, etc.

### Hydrogel Matrix Systems of the Invention

A two-component hydrogel matrix system is described, which contains two primary components that comprise: (1) chemically modified hyaluronic acid (HA) comprising an aldehyde or benzaldehyde containing side group, and a bicyclononyne containing side group, and (2) chemically modified elastin-like protein (ELP) comprising a hydrazine containing side group and an azide containing side group. Mixing the two modified biopolymers together induces the formation of dynamic bonds between the aldehyde or benzaldehyde containing side group and the hydrazine containing side group (also known as hydrazone bonds) and static bonds between the bicyclononyne containing side group and the azide containing side group (referred herein as spot welds) resulting in the formation of a hydrogel network. Selection of the ratio between (1) and (2) allows tuning of critical variables of the hydrogel, including, for example, matrix stiffness, matrix stress relaxation rate, and cell-adhesive-ligand concentration and identity. These variables can be independently and quantitatively defined.

In some embodiments, the hyaluronic acid component is a single component comprising chemically modified hyaluronic acid that comprises a benzaldehyde or aldehyde side group and a bicyclononyne containing side group. In some embodiments, the hyaluronic acid component is a two components where the first component comprises chemically modified hyaluronic acid that comprises a benzaldehyde or aldehyde side group and the second component comprises chemically modified hyaluronic acid that comprises a bicyclononyne containing side group. The specific ratio of the benzaldehyde and the aldehyde side groups in the final hydrogel formulation controls the stress relaxation variable. A key feature of native extracellular matrices is their ability to undergo stress relaxation due to their physical crosslinks, which can be easily remodeled. Compositions comprising a greater percentage of hyaluronic acid modified with an aldehyde containing side group, increase the average kinetic exchange rate of the gel, leading to a faster stress-relaxation rate. A greater percentage of hyaluronic acid modified with a benzaldehyde side group decreases the average kinetic exchange rate of the gel, leading to slower stress-relaxation rate. Modifications to stress-relaxation rate can be achieved independently of matrix ligand composition and stiffness. The ratio of HA-benzaldehyde to HA-aldehyde may be pre-selected for a hydrogel of interest, usually ranging from about 100:0 to 0:100, for example at a ratio from about 95:5, 90:10, 75:25, 50:50, 25:75, etc. The final concentration of chemically modified HA in the hydrogel may range from about 0.5 wt % to 3 wt %, for example at 0.5 wt %, 1.0 wt %, 1.5 wt %, 2.0 wt %, 2.5 wt %, or 3 wt %.

When the hyaluronic acid component is two components, the first HA and second HA component may be in a specific ratio. The ratio of the first HA component to the second HA component may be in a range from at least about 100:1 to at least about 1:1. For instance, the ratio of the first HA to the second HA component may be at least about 100:1, at least about 95:1, at least about 90:1, at least about 85:1, at least about 80:1, at least about 75:1, at least about 70:1, at least about 65:1, at least about 60:1, at least about 55:1, at least about 50:1, at least about 45:1, at least about 40:1, at least about 35:1, at least about 30:1, at least about 25:1, at least about 20:1, at least about 15:1, at least about 10:1, at least about 5:1, or at least about 1:1.

The ELP component comprises a recombinant sequence of elastin-like sequences optionally interspersed with cell-adhesive sequences. To engage in crosslinking with chemically modified HA, the ELP is chemically modified to comprise a hydrazine group and an azide group. In some embodiments, the ELP component is a single component comprising a chemically modified ELP to comprise a hydrazine group and an azide group. In some embodiments, the ELP component is two components where the first component comprises a chemically modified ELP to comprise a hydrazine group and the second component comprises a chemically modified ELP to comprise a an azide group The optional cell-adhesive sequence within the ELP may be selected from an integrin-binding, fibronectin-based, extended RGD sequence, a scrambled RGD sequence, a cell-adhesive sequence derived from collagen type I, e.g. (SEQ ID NO:3) DGEA, a cell adhesive sequence derived from tenascin, e.g. (SEQ ID NO:4) PLAEIDGIELTY, (SEQ ID NO:5) VFDNFVLK, etc.; a cell adhesive sequence derived from laminin, e.g. (SEQ ID NO:6) IKVAV, (SEQ ID NO:7) YIGSR, etc.; a cell adhesive sequence derived from cadherin, e.g. (SEQ ID NO:8) HAVDI, (SEQ ID NO:9) HAVDIHAVDI; and the like. For example, SEQ ID NO:1 and SEQ ID NO:2 are ELPs with an RGD sequence, and a scrambled RGD sequence, respectively.

Sequences for an elastin-like region may include, for example,

(SEQ ID NO: 20)
(VPGIG)₂(VPGKG)(VPGIG)₂;
or
(SEQ ID NO: 21)
(VPGIG)(VPGKG)(VPGYG)(VPGIG)(VPGKG)(VPGIG).

When the ELP component is two components, the first ELP and second ELP component may be in a specific ratio. The ratio of the first ELP component to the second ELP component may be in a range from at least about 100:1 to at least about 1:1. For instance, the ratio of the first ELP to the second ELP component may be at least about 100:1, at least about 95:1, at least about 90:1, at least about 85:1, at least about 80:1, at least about 75:1, at least about 70:1, at least about 65:1, at least about 60:1, at least about 55:1, at least about 50:1, at least about 45:1, at least about 40:1, at least about 35:1, at least about 30:1, at least about 25:1, at least about 20:1, at least about 15:1, at least about 10:1, at least about 5:1, or at least about 1:1. The final concentration of chemically modified ELP in the hydrogel may range from about 0.5 wt % to 3 wt %, for example at 0.5 wt %, 1.0 wt %, 1.5 wt %, 2.0 wt %, 2.5 wt %, or 3 wt %.

The cell-adhesive sequence concentration of the hydrogel can be varied by adjusting the ratio of ELP comprising an RGD motif, to ELP lacking an RGD motif or comprising scrambled or non-RGD cell-adhesive motifs, as disclosed above. The ratio may be pre-selected for a hydrogel of interest, usually ranging from about 100:0 to 0:100, for example from about 75:25, 50:50, 25:75, 10:90. For some applications, a HELP hydrogel comprises from about 0.25 mM RGD up to about 1.5 mM RGD, e.g. about 0.25 mM, 0.5 mM, 0.75 mM, 1 mM, 1.25 mM, 1.5 mM.

In addition to the two polymers, the hydrogel matrix system may also comprise a solvent in which the two polymers are solubilized in. A range of different solvents may be used. Solvents that find use in the present disclosure include, without limitation, water, saline, cell culture medium, etc. The hydrogel matrix system may also further comprise bioactive agents. A range of different bioactive agents may be used in the hydrogel matrix system. Bioactive agents that find use in the present disclosure include, without limitation, growth factors, small molecules, chemicals, proteins, DNA, mRNA, drugs, cells, stem cells, organoids, etc. The hydrogel matrix system spontaneously forms upon mixing of the two components (i.e. the chemically modified HA as described above and the chemically modified ELP as described above).

When the bioactive agent is a growth factor, the growth factor of may include, without limitation, insulin, glucagon, growth hormone (GH), parathyroid hormone (PTH), growth hormone releasing factor (GHRF), follicle stimulating hormone (FSH), luteinizing hormone (LH), human chorionic gonadotropin (hCG), vascular endothelial growth factor (VEGF), angioproteinetins, angiostatin, granulocyte colony stimulating factor (GCSF), erythroproteinetin (EPO), connective tissue growth factor (CTGF), basic fibroblast growth factor (bFGF), acidic fibroblast growth factor (aFGF), epidermal growth factor (EGF), transforming growth factor .alpha. (TGFa), platelet-derived growth factor (PDGF), insulin growth factors I and II (IGF-1 and IGF-11), any one of the transforming growth factor 13-superfamily, including TGFI3, activins, inhibins, or any of the bone morphogenic proteins (BMP) including BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Matrix stiffness is determined by the concentration and ratio of hydrazine to aldehyde and benzaldehyde side groups (dynamic bonds) and the concentration of bicyclononyne to azide side groups (static bonds). The ratio of the hydrazine to aldehyde and benzaldehyde side groups may be varied, e.g. from about 1:3, about 1:2, about 1.5:1, about 1.25:1, about 1:1, about 1:1.25, about 1:1.5, about 1:2, about 1:3, etc. The ratio of the bicyclononyne to azide reactive groups may be varied, e.g. from about 1:3, about 1:2, about 1.5:1, about 1.25:1, about 1:1, about 1:1.25, about 1:1.5, about 1:2, about 1:3, etc.

The ratio of the dynamic bonds to static bonds may have an impact on the matrix stiffness or may not alter matrix stiffness depending on the ratio of dynamic to static bonds. The ratio of dynamic to static bonds may be in a range that is at least about 30:1, about 14:1, about 7:1, about 5:1, to at least about 3:1. For instance, the ratio of dynamic to static bonds in the hydrogel matrix system may be in a ratio of at least about 30:1, at least about 28:1, at least about 26:1, at least about 24:1, at least about 22:1, at least about 20:1, at least about 18:1, at least about 16:1, at least about 14:1, at least about 12:1, at least about 10:1, at least about 9:1, at least about 8:1, at least about 7:1, at least about 6:1, at least about 5:1, at least about 4:1, at least about 3.5:1, at least about 3:1, etc. Depending on the specific ratio of dynamic to static bonds or the ratio of hydrazine to aldehyde and benzaldehyde side groups, the matrix stiffness may vary from at least about 100 Pa to at least about 15 kPa. The ratio of dynamic bonds and static bonds can be altered through three variables: (1) number of hydrazine and azide groups per ELP molecule, (2) number of aldehyde or benzaldehyde and bicyclononyne groups per HA molecule, and (3) the blending of ELP and HA.

The presence of static bonds within a hydrogel matrix system comprising dynamic bonds may provide a number of benefits. In some embodiments, the presence of static bonds within a hydrogel matrix system comprising dynamic bonds does not affect the overall stiffness of the hydrogel matrix system as compared to a hydrogel matrix system comprising only dynamic bonds while increasing the stability of the hydrogel matrix system. In some embodiments, the presence of static bonds within a hydrogel matrix system comprising dynamic bonds does not inhibit the shear thinning behavior of the hydrogel matrix system as compared to a hydrogel matrix system comprising only dynamic bonds while increasing the stability of the hydrogel matrix system.

The presence of static bonds within a hydrogel matrix system comprising dynamic bonds increases the stability of the hydrogel matrix system. The stability of the hydrogel matrix system may be measured as the resistance or delay of erosion of the hydrogel matrix system. The erosion of the hydrogel matrix system is measured by the amount of ELP polymer remaining at a given time point. The presence of static bonds within a hydrogel matrix system comprising dynamic bonds may increase the amount of time that the hydrogel matrix system is able to retain at least 50% of the ELP within the hydrogel matrix system. The amount of time may be a range of different times. For instance, the presence of static bonds within a hydrogel matrix system comprising dynamic bonds may retain at least 50% of the ELP within the hydrogel matrix system for at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, or greater than about 30 days.

The presence of static bonds within a hydrogel matrix system comprising dynamic bonds may increase the amount of time that the hydrogel matrix system is able to retain at least 60% of the ELP within the hydrogel matrix system. The amount of time may be a range of different times. For instance, the presence of static bonds within a hydrogel matrix system comprising dynamic bonds may retain at least 60% of the ELP within the hydrogel matrix system for at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, or greater than about 30 days.

The presence of static bonds within a hydrogel matrix system comprising dynamic bonds may increase the amount of time that the hydrogel matrix system is able to retain at least 70% of the ELP within the hydrogel matrix system. The amount of time may be a range of different times. For instance, the presence of static bonds within a hydrogel matrix system comprising dynamic bonds may retain at least 70% of the ELP within the hydrogel matrix system for at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, or greater than about 30 days.

In another aspect of the invention, the matrix is extruded through a syringe needle or a catheter to form a solid disc structure. The solid disc structure maintains its structural stability after release from the printing support bath. After extrusion, the solid disc structure maintains its structural stability. Cells encapsulated within the HELP matrix can be extruded in this manner for use as a bio-ink for 3D bioprinting; for injectable regenerative medicine therapy; etc. A shear-thinning material with a fracture stress below 2,000 Pa is injectable by hand force. The fracture stress can be adjusted by changing three variables: (1) the molecular weight of the HA, usually with MW below 100 kDa, (2) the kinetics of the hydrazone bond, with the fast exchange kinetics of the hydrazine-aldehyde preferred over the slower exchange kinetics of the hydrazine-benzaldehyde reaction for this purpose, and (3) the overall polymer concentration, usually with a final concentration of from about 0.5-3 wt % of ELP and from about 0.5-3 wt % of HA.

### Kits of the Invention

Also provided are kits for generating hydrogels comprising the hydrogel matrix system of the present disclosure. In general, the subject kits comprise the hydrogel matrix system or components thereof, e.g., as described above. In some cases, the kit further comprises a solvent in which to suspend the chemically modified HA and ELP as described above. The kit may also contain a bioactivate agent as described above.

The hydrogel matrix system comprises two primary components that comprise: (1) chemically modified hyaluronic acid (HA) comprising an aldehyde or benzaldehyde containing side group, and a bicyclononyne containing side group, and (2) chemically modified elastin-like protein (ELP) comprising a hydrazine containing side group and an azide containing side group.

In some embodiments, the hyaluronic acid component is a single component comprising chemically modified hyaluronic acid that comprises a benzaldehyde or aldehyde side group and a bicyclononyne containing side group. In some embodiments, the hyaluronic acid component is two components where the first component comprises chemically modified hyaluronic acid that comprises a benzaldehyde or aldehyde side group and the second component comprises chemically modified hyaluronic acid that comprises a bicyclononyne containing side group The specific ratio of the benzaldehyde and the aldehyde side groups in the final hydrogel formulation controls the stress relaxation variable. A key feature of native extracellular matrices and EHS matrices is their ability to undergo stress relaxation due to their physical crosslinks, which can be easily remodeled. Compositions comprising a greater percentage of hyaluronic acid modified with an aldehyde containing side group, increase the average kinetic exchange rate of the gel, leading to a faster stress-relaxation rate. A greater percentage of hyaluronic acid modified with a benzaldehyde group decreases the average kinetic exchange rate of the gel, leading to slower stress-relaxation rate. Modifications to stress-relaxation rate can be achieved independently of matrix ligand composition and stiffness. The ratio of HA-benzaldehyde to HA-aldehyde may be pre-selected for a hydrogel of interest, usually ranging from about 100:0 to 0:100, for example at a ratio from about 95:5, 90:10, 75:25, 50:50, 25:75, etc.

When the hyaluronic acid component is two components, the first and second component may be in a specific ratio. The ratio of the first component to the second component may be in a range from at least about 100:1 to at least about 1:1. For instance, the ratio of the first to the second component may be at least about 100:1, at least about 95:1, at least about 90:1, at least about 85:1, at least about 80:1, at least about 75:1, at least about 70:1, at least about 65:1, at least about 60:1, at least about 55:1, at least about 50:1, at least about 45:1, at least about 40:1, at least about 35:1, at least about 30:1, at least about 25:1, at least about 20:1, at least about 15:1, at least about 10:1, at least about 5:1, or at least about 1:1.

The ELP component comprises a recombinant sequence of elastin-like sequences optionally interspersed with cell-adhesive sequences. To engage in crosslinking with chemically modified HA, the ELP is chemically modified to comprise a hydrazine group and an azide group. In some embodiments, the ELP component is a single component comprising a chemically modified ELP to comprise a hydrazine group and an azide group. In some embodiments, the ELP component is two components where the first component comprises a chemically modified ELP to comprise a hydrazine group and the second component comprises a chemically modified ELP to comprise an azide group The optional cell-adhesive sequence within the ELP may be selected from an integrin-binding, fibronectin-based, extended RGD sequence, a scrambled RGD sequence, a cell-adhesive sequence derived from collagen type I, e.g. (SEQ ID NO:3) DGEA, a cell adhesive sequence derived from tenascin, e.g. (SEQ ID NO:4) PLAEIDGIELTY, (SEQ ID NO:5) VFDNFVLK, etc.; a cell adhesive sequence derived from laminin, e.g. (SEQ ID NO:6) IKVAV, (SEQ ID NO:7) YIGSR, etc.; a cell adhesive sequence derived from cadherin, e.g. (SEQ ID NO:8) HAVDI, (SEQ ID NO:9) HAVDIHAVDI; and the like. For example, SEQ ID NO:1 and SEQ ID NO:2 are ELPs with an RGD sequence, and a scrambled RGD sequence, respectively.

When the ELP component is two components, the first and second component may be in a specific ratio. The ratio of the first component to the second component may be in a range from at least about 100:1 to at least about 1:1. For instance, the ratio of the first to the second component may be at least about 100:1, at least about 95:1, at least about 90:1, at least about 85:1, at least about 80:1, at least about 75:1, at least about 70:1, at least about 65:1, at least about 60:1, at least about 55:1, at least about 50:1, at least about 45:1, at least about 40:1, at least about 35:1, at least about 30:1, at least about 25:1, at least about 20:1, at least about 15:1, at least about 10:1, at least about 5:1, or at least about 1:1.

In addition to the two polymers, the hydrogel matrix system may also comprise a solvent in which the two polymers are solubilized in. A range of different solvents may be used. Solvents that find use in the present disclosure include, without limitation, water, saline, cell culture medium, etc. The hydrogel matrix system may also further comprise bioactive agents. A range of different bioactive agents may be used in the hydrogel matrix system. Bioactive agents that find use in the present disclosure include, without limitation, growth factors, small molecules, chemicals, proteins, DNA, mRNA, drugs, stem cells, organoids, etc. The hydrogel matrix system spontaneously forms upon mixing of the two components (i.e. the chemically modified HA as described above and the chemically modified ELP as described above).

A subject kit may include any combination of the components of the hydrogel matrix system, e.g. the chemically modified HA, the chemically modified ELP, the solvent, the bioactive agent. The components of a subject kit can be present as a mixture or can be separate entities. In some cases, components are present as a lyophilized mixture. In some cases, the components are present as a liquid mixture. Components of a subject kit can be in the same or separate containers, in any combination.

The subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a remote site.

### Experimental

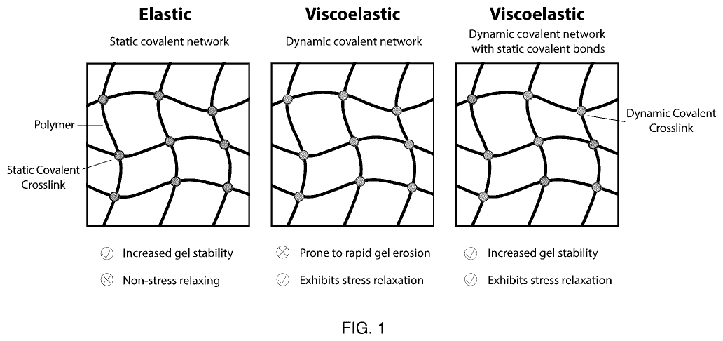

Viscoelasticity is a desirable material property for biomaterials, as it allows for many gels to be injectable and for cells to more easily migrate into/through, proliferate, spread, and deposit matrix within the gel. However, most viscoelastic gels suffer from poor stability because they are prone to rapid gel erosion, since the crosslinks within the polymer network are dynamic and reversible (blue; FIG. 1 middle panel). Elastic gels offer high stability because they have static covalent crosslinks with the polymer network, however, do not have stress relaxation properties (FIG. 1 left panel). This problem was overcome by creating a polymer network that includes both static (gray) and reversible (blue) crosslinks (FIG. 1 right panel), so that the material is viscoelastic while also being more stable.

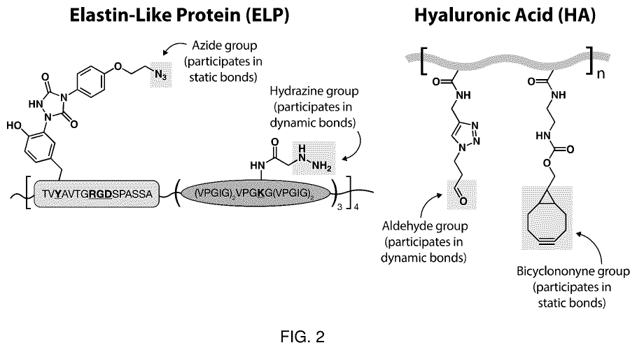

FIG. 2 shows a chemical schematic of one specific embodiment of the two components used to formulate the hydrogel invention. The hydrogel matrix system is composed of two different polymers. Each polymer has been modified through bioconjugation to display two different types of reactive chemical functional groups. The first polymer is a recombinant elastin-like protein (ELP), that is modified to display azides and hydrazines. The second polymer is a recombinant hyaluronic acid (HA) that has been modified to display aldehydes and bicyclononynes. The azides and bicyclononynes react to form stable covalent bonds, which is called “spot welds”. Simultaneously, the hydrazines and aldehydes react to form reversible, dynamic covalent bonds. The two polymers are solubilized in an aqueous solvent (e.g., water, saline, cell culture medium) in the presence or absence of bioactive agents (e.g. growth factors, drugs, cells) and stirred together. The hydrogel network spontaneously forms upon mixing of the two components.

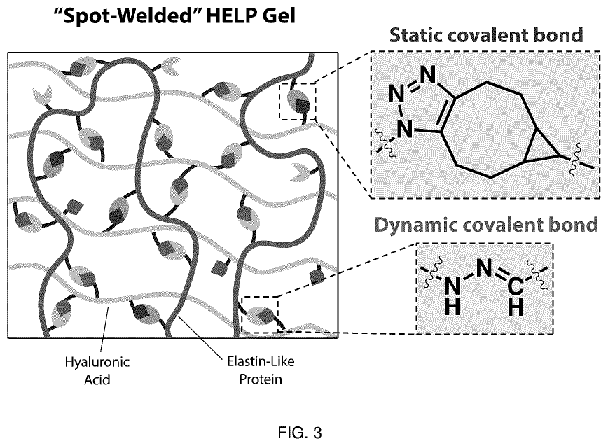

FIG. 3 shows one specific embodiment of the hydrogel invention with exemplar static and dynamic crosslink bonds.

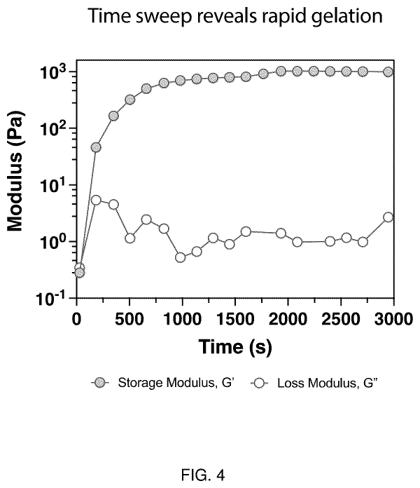

As shown in FIG. 4, gelation of the hydrogel matrix system occurs spontaneously after mixing at time zero, as characterized by the G′ storage modulus value being much larger than the G″ loss modulus value. These data are obtained for an exemplar hydrogel formulation of 1 wt % ELP with hydrazine and azide groups and 1 wt of HA with aldehyde and bicyclononyne groups, where the ratio of dynamic to static bonds is 3.5:1.

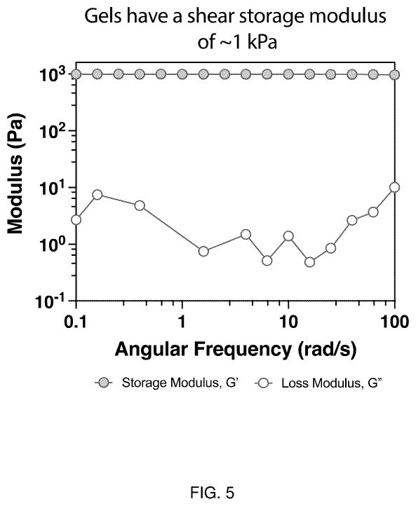

As shown in FIG. 5, the hydrogel matrix system has a large, stable, plateaus storage modulus region across a range of frequencies, which is common for elastic hydrogels formed from only static covalent crosslinks, but less common for viscoelastic hydrogels. While the gel shown here has a stiffness ˜1 kPa, the final gel stiffness can be controlled by altering the gel formulation to range between 100 Pa-15 kPa. These data are obtained for an exemplar hydrogel formulation of 1 wt % ELP with hydrazine and azide groups and 1 wt of HA with aldehyde and bicyclononyne groups, where the ratio of dynamic to static bonds is 3.5:1.

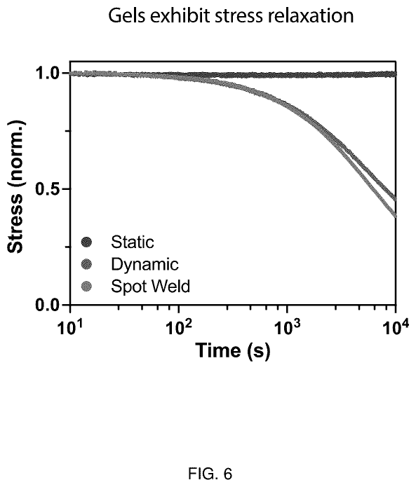

As shown in FIG. 6, the hydrogel matrix system (labeled as “spot weld”) has a stress relaxation rate, which is common for viscoelastic hydrogels (such as the gel labeled “dynamic” which only has dynamic covalent crosslinks; also known as dynamic bonds) but is not observed in elastic hydrogels (such as the gel labeled “static” which only has static covalent crosslinks also known as dynamic bonds) Thus, the hydrogel matrix system combines the desired mechanical properties of both static covalent gels and dynamic covalent gels. The “spot weld” data are obtained for an exemplar hydrogel formulation of 1 wt % ELP with hydrazine and azide groups and 1 wt of HA with aldehyde and bicyclononyne groups, where the ratio of dynamic to static bonds is 3.5:1. The control “dynamic” data are obtained from a hydrogel formulation of 1 wt % ELP with hydrazine groups and 1 wt of HA with aldehyde groups, where only dynamic bonds can be formed. The control “static” data are obtained from a hydrogel formulation of 1 wt % ELP with azide groups and 1 wt of HA with bicyclononyne groups, where only static bonds can be formed.

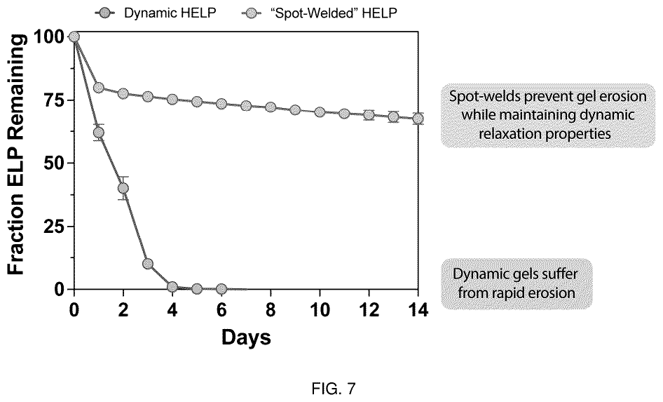

FIG. 7 shows that typical viscoelastic gels made from only dynamic crosslinks (dynamic HELP) erode very rapidly. Here erosion is quantified by measuring how much of the ELP polymer is left in the hydrogel at different times. The hydrogel has completed degraded by day 4. In contrast, combining the dynamic crosslinks with the spot-welds of static covalent crosslinks into the gel (the hydrogel matrix system; also known as spot-welded HELP), it is seen that the gel can persist up to two weeks and has much slower erosion. The “Spot-Welded HELP” data are obtained for an exemplar hydrogel formulation of 1 wt % ELP with hydrazine and azide groups and 1 wt of HA with aldehyde and bicyclononyne groups, where the ratio of dynamic to static bonds is 3.5:1. The control “Dynamic HELP” data are obtained from a hydrogel formulation of 1 wt % ELP with hydrazine groups and 1 wt of HA with aldehyde groups, where only dynamic bonds can be formed.

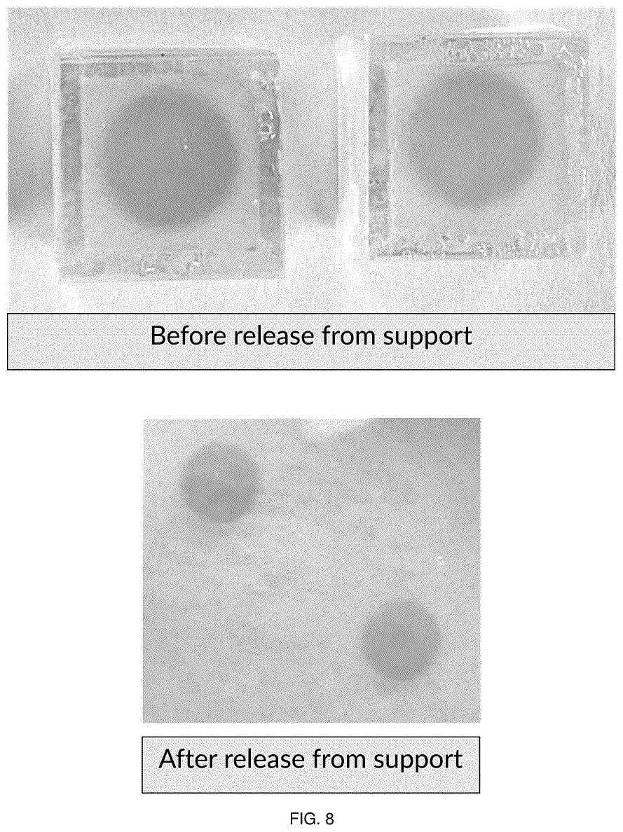

FIG. 8 shows that the hydrogel matrix system can be formulated to still be injectable and ejectable. Here it is demonstrate that the hydrogel matrix system can be used as an extrusion ink in a 3D bioprinter. The gel is extruded through a 27-G needle to form a solid disc structure within a printing support bath. Here the printing support bath is colloidal gelatin microspheres as an exemplary material, and the support bath could also be other viscoelastic media that support embedded bioink printing. The disc maintains its structural stability after release from the printing support bath. This is very difficult to achieve with inks that only have reversible, dynamic crosslinks, since the structure is not stable. These data are obtained for an exemplar hydrogel formulation of 1 wt % ELP with hydrazine and azide groups and 1 wt of HA with aldehyde and bicyclononyne groups, where the ratio of dynamic to static bonds is 3.5:1.

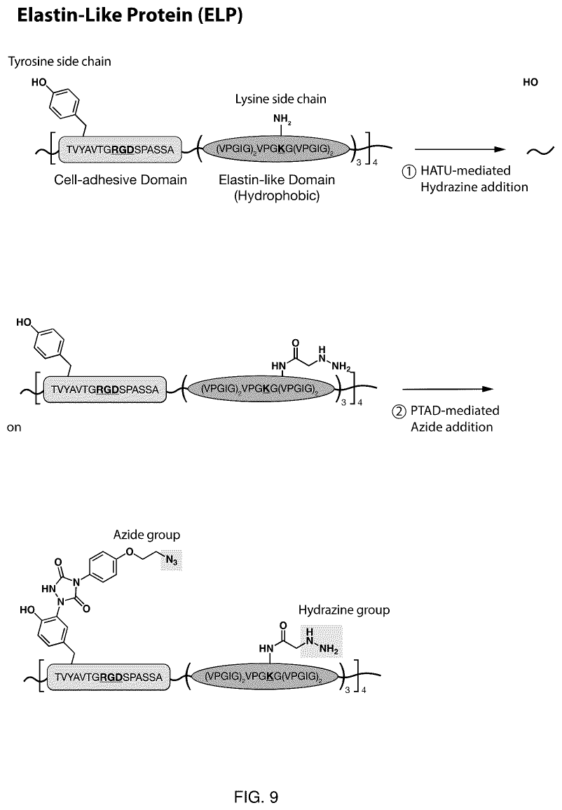

As shown in FIG. 9, the azide group and the hydrazine groups are added to the elastin-like protein (ELP) through a number of synthesis steps. As a brief, non-exhaustive description, first, the hydrazine group is added to ELP through HATU-mediated addition. In the second step, the azide group is added through PTAD-mediated azide addition resulting in an ELP comprising both an azide and a hydrazine side group.

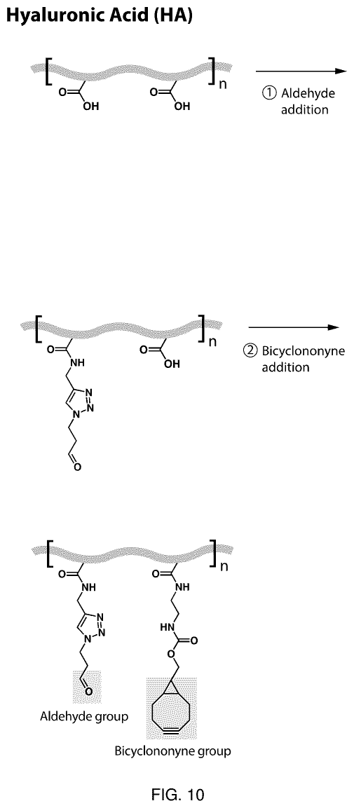

FIG. 10 shows the aldehyde and the bicyclononyne groups are added to the hyaluronic acid (HA) through a number of synthesis steps. As a brief, non-exhaustive description, first, the aldehyde group is added to HA. In the second step, the bicyclononyne group is added resulting in an HA comprising both an aldehyde and a bicyclononyne side group.

Sequences

SEQ ID NO: 1,
LQ(LDASTVYAVGRGDSPASSA[(VPGIG)₂VPGKG(VPGIG)₂]₃)₄
SEQ ID NO: 2,
LQ(LDASTVYAVGRDGSPASSA[(VPGIG)₂VPGKG(VPGIG)₂]₃)₄.
(SEQ ID NO: 3)
DGEA
SEQ ID NO: 4)
PLAEIDGIELTY
(SEQ ID NO: 5)
VFDNFVLK
(SEQ ID NO: 6)
IKVAV,
(SEQ ID NO: 7)
YIGSR
(SEQ ID NO: 8)
HAVDI,
(SEQ ID NO: 9)
HAVDIHAVDI

| ECM- | | |
|---|---|---|
| derived | | |
| Protein | Cell-adhesive domain | Elastin-Like Region |
| h- | (SEQ ID NO: 10) | (SEQ ID NO: 20) |
| Fibronectin | TVYAVTGRGDSPASSAA | (VPGIG)₂ |
| | | (VPGKG)(VPGIG)₂ |
| Laminin | (SEQ ID NO: 11) | (SEQ ID NO: 20) |
| (β1 | VSDPGYIGSRSDDSASASAA | (VPGIG)₂(VPGKG)(VPGIG)₂ |
| chain) | | |
| Laminin | (SEQ ID NO: 22) | (SEQ ID NO: 21) |
| (α1 | ARKQAASIKVAVSADRASA | (VPGIG)(VPGKG)(VPGYG) |
| chain) | | (VPGIG)(VPGKG)(VPGIG) |
| Collagen | (SEQ ID NO: 12) | (SEQ ID NO: 20) |
| 1 | VGPAGGDGEAGAQGPP | (VPGIG)₂(VPGKG)(VPGIG)₂ |
| h- | (SEQ ID NO: 13) | (SEQ ID NO: 20) |
| Tenascin- | SGSGGSGGPLAEIDGIELTY | (VPGIG)₂(VPGKG)(VPGIG)₂ |
| C | GGSGGSGS | |
| h- | (SEQ ID NO: 14) | (SEQ ID NO: 21) |
| Tenascin- | SGSGGSGGLDVFDNFVLKGGSG | (VPGIG)(VPGKG)(VPGYG) |
| C | GSGS | (VPGIG)(VPGKG)(VPGIG) |
| h- | (SEQ ID NO: 15) | (SEQ ID NO: 20) |
| Tenascin- | SGSGGSGGLDVFDNFVLGGSGG | (VPGIG)₂(VPGKG)(VPGIG)₂ |
| C | SGS | |
| N- | (SEQ ID NO: 16) | (SEQ ID NO: 20) |
| cadherin | SGSGGSGGHAVDIGGSGGSGS | (VPGIG)₂(VPGKG)(VPGIG)₂ |
| N- | (SEQ ID NO: 17) | (SEQ ID NO: 20) |
| cadherin | SGSGGSGGHAVDINGHAVDIG | (VPGIG)₂(VPGKG)(VPGIG)₂ |
| | GSGGSGS | |
| Non- | (SEQ ID NO: 18) | (SEQ ID NO: 20) |
| binding | SGSGGSGGADHIVGGSGGSGS | (VPGIG)₂(VPGKG)(VPGIG)₂ |
| Non- | (SEQ ID NO: 19) | (SEQ ID NO: 20) |
| binding | TVYAVTGRDGSPASSAA | (VPGIG)₂(VPGKG)(VPGIG)₂ |

(SEQ ID NO: 23) GGGGS
(SEQ ID NO: 24) GSTSGSGKSSEGKG
(SEQ ID NO: 25) VPGIG
(SEQ ID NO: 26) VPGKG
(SEQ ID NO: 27) VPGYG

What is claimed is:

1. A two-component hydrogel matrix system comprising:
a first component comprising a defined ratio of hyaluronic acid (HA) modified to comprise an aldehyde side group, HA modified to comprise a benzaldehyde side group and HA modified to comprise a bicyclononyne side group; and
a second component comprising an elastin-like protein (ELP) modified to comprise a hydrazine side group and an azide side group;
wherein cross-links between the first component and the second component are formed to generate a hydrogel upon mixing.

2. The system of claim 1, wherein matrix stiffness is independently tuned by varying the ratio of the side groups present on the first component, to side groups present on the second component.

3. The system of claim 1, wherein the ratio of reactant groups is varied by one or more of:
specifying the number of side groups on HA; specifying the number of side groups on ELP; and
specifying the ratio of HA:ELP.

4. The system of claim 1, wherein matrix stress relaxation rate is independently tuned by varying the ratio of hyaluronic acid modified to comprise an aldehyde side group, and hyaluronic acid modified to comprise a benzaldehyde side group in the first component.

5. The system of claim 1, wherein the ratio of hyaluronic acid modified to comprise a pendant aldehyde, and hyaluronic acid modified to comprise a pendant benzaldehyde is from 100:0 to 0:100.

6. The system of claim 1, wherein the ratio of dynamic bonds to static bonds between the first and the second component is between 30:1 and 3:1.

7. The system of claim 1, wherein the static bonds between the first and second component do not alter the stiffness of the system relative to a system in the absence of the static bonds.

8. The system of claim 1, wherein the static bonds between the first and second component do not inhibit the shear thinning behavior of the system relative to a system in the absence of the static bonds.

9. The system of claim 1, wherein the static bonds between the first and second component retain at least 60% of the ELP within the hydrogel matrix system from at least 14 days.

10. The system of claim 1, wherein the static bonds between the first and second component retain at least 70% of the ELP within the hydrogel matrix system from at least 14 days.

11. The system of claim 1, wherein the hydrogel can be extruded though a syringe or catheter.

12. The system of claim 1, further comprising a solvent.

13. The system of claim 12, wherein the solvent is selected from the group consisting of water, saline, and cell culture medium.

14. The system of claim 1, further comprising a bioactive agent.

15. The system of claim 14, wherein the bioactive agent is selected from the group consisting of a growth factor, a small molecule, a chemical, a protein, a DNA, a mRNA, a drug, a cell, a stem cell, and an organoid.

16. A hydrogel formed from the system of claim 1.

17. A cell culture medium comprising the hydrogel of claim 16.

18. A kit, the kit comprising:
(i) a first component comprising a defined ratio of hyaluronic acid (HA) modified to comprise an aldehyde side group, HA modified to comprise a benzaldehyde side group and HA modified to comprise a bicyclononyne side group and
(ii) a second component comprising an elastin-like protein (ELP) modified to comprise a hydrazine side group and an azide side group.

19. The kit of claim 18, further comprising a solvent selected from the group consisting of water, saline, and cell culture medium.

20. The kit of claim 19, further comprising a comprising a bioactive agent selected from the group consisting of a growth factor, a small molecule, a chemical, a protein, a DNA, a mRNA, a drug, a cell, a stem cell, and an organoid.
